(19) United States
(12) Patent Application Publication
(10) Pub. No.: US 2009/0203938 A1
(43) Pub. Date: Aug. 13, 2009

(54) USE OF PHOSPHORIC ESTERS AS DEPOSIT CONTROL AGENTS DURING THE SYNTHESIS, PURIFICATION OR REGENERATION OF (METH)ACRYLIC MONOMERS

(75) Inventors: Jean-Francois Croizy, Carling (FR); Michel Fauconet, Valmont (FR); Jean-Michel Paul, Metz (FR); Rene Bonnaz, Bougival (FR)

Correspondence Address: ARKEMA INC.;PATENT DEPARTMENT - 26TH FLOOR, 2000 MARKET STREET, PHILADELPHIA, PA, 19103-3222, US

(73) Assignee: Arkema France, Puteaux (FR)

(21) Appl. No.: 11/577,072

(22) PCT Filed: Oct. 7, 2005
(86) PCT No.: PCT/FR2005/002480

§ 371 Date: Apr. 11, 2007

(30) Foreign Application Priority Data
Oct. 11, 2004 (FR) 0410687

(51) Int. Cl. C07C 51/44; C07C 51/42

(52) U.S. Cl. 562/600

(57) ABSTRACT

The compounds of formula (I) are provided as deposition inhibitors which prevent the deposition of compounds of polymeric nature or resulting from addition reactions to (meth)acrylic double bonds during operations for the synthesis or purification or regeneration of (meth)acrylic monomers.

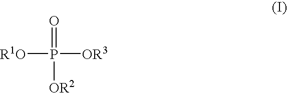

R¹ represents a C₃-C₃₀ alkyl radical, an aryl radical or an alkylaryl radical, it being possible for these radicals to be interrupted by or connected to the oxygen of the molecule via a —(OR⁴)ₒ— chain where the R⁴ groups each independently represent an ethylene, propylene or butylene chain and o is an integer from 1 to 50;
R² represents R¹, a hydrogen atom or a counterion; and
R³ represents a hydrogen atom or a counterion.

The present invention belongs to the field of the synthesis, purification and regeneration of (meth)acrylic monomers, such as acrylic acid and methacrylic acid as well as C₁-C₈ alkyl acrylates and methacrylates. More particularly, the present invention relates to the suppression or at least the limitation of the phenomena of fouling due to the deposition of insoluble polymers and/or of insoluble heavy products in plants used for the abovementioned synthesis, purification or regeneration operations.

Mention may in particular be made, as reactions for the synthesis of (meth)acrylic monomers, of:
the gas-phase catalytic oxidation of propylene or propane to manufacture acrylic acid;
the catalytic oxidation of isobutene or tert-butanol to generate methacrylic acid;
the synthesis of methacrylamide sulphate from acetone cyanohydrin, methanol and sulphuric acid to produce methyl methacrylate;
the production of (meth)acrylic esters from (meth)acrylic acid and alcohols.

It is in fact well known that one of the problematic points in the manufacture of (meth)acrylic monomers arises from the fact that these compounds are relatively unstable and easily change in the direction of the formation of polymers. This change, caused by a radical reaction promoted by the effect of the temperature, is thus particularly easy in the stages of synthesis and of purification of these monomers, for example during distillation operations. The usual consequence of this process is the deposition, in the items of equipment of a plant, of solid polymers which end up causing blockages and make it necessary to shut down the plant in order to clean it, which proves to be difficult and expensive in unproductive downtime.

Polymerization inhibitors are added to the streams in order to reduce these disadvantages. Numerous polymerization inhibitors are used conventionally in processes for the purification of (meth)acrylic monomers. Mention may be made, among them, of phenolic compounds, phenothiazine and its derivatives, manganese salts, thiocarbamates and dithiocarbamates, N-oxyl compounds, amino compounds and nitroso compounds, examples of these various compounds being indicated later.

A large majority of polymerization inhibitors are compounds which are heavier than the (meth)acrylic monomers to be purified. In the distillation stages, due to their low volatility, they are only to a very slight extent entrained in the vapour phases in equilibrium with the liquid phases of the rectification plates and consequently cannot carry out their role of protecting the monomer against polymerization in the upper parts of the columns. For this reason, these inhibitors are generally introduced at the level of the top items of equipment of columns, condensers, and the like, which can be the site of a liquid-vapour equilibrium resulting in the condensation of streams rich in (meth)acrylic monomer.

The effectiveness of these polymerization inhibitors, used alone or as a mixture, is generally enhanced when they are used in combination with the introduction of oxygen or of a gas stream comprising oxygen at the column bottom. These polymerization inhibitors can be introduced as they are, for example when they are liquid, or in solution in a solvent and, preferably, in solution in the monomer itself.

Despite the use of polymerization inhibitors, the operations for the purification of monomers are often accompanied by the formation of polymers, which are soluble or insoluble in the medium according to the nature of the monomer and the composition of the medium. In the most troublesome cases, the polymer formed is insoluble in the medium and precipitates in the form of a solid deposit.

The precipitation of the polymer from the medium occurs in particular when the length of the polymer chain is sufficient or when polyfunctional compounds generate bridges between certain units of the polymer by a process known under the name of crosslinking.

Another source of fouling of the plants by solid deposition is the presence of heavy compounds generated during the reaction stage of synthesis of the monomer.

For example, the manufacture of (meth)acrylic monomers is accompanied by the formation of compounds resulting from a Michael addition reaction of a molecule comprising a labile hydrogen atom to the double bond of a (meth)acrylic compound:

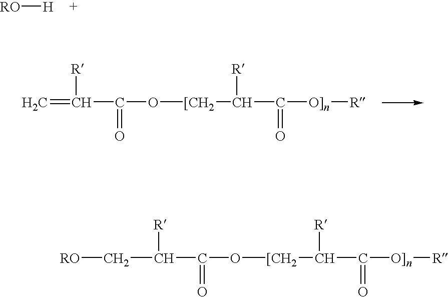

with:
R=H, C₁-C₈ alkyl or CH₃—C(═O)—
R′=H or methyl
R″=H or C₁-C₈ alkyl
n≧0.

These addition compounds may, depending on the medium in which they are present and their molar masses (proportional to the number of units n), be insoluble in this medium.

The formation of insoluble polymers or of insoluble heavy compounds can take place rapidly at the spot itself where the polymerization process or the Michael addition reaction are initiated, if the polymer or the heavy compound are insoluble in the medium, or can take place during a stage of the purification process in which these products, dissolved beforehand, become insoluble by modification of the composition of the original medium, for example during an operation in which the heavy compounds are concentrated by separation of the light compounds or during the removal of a polar component responsible for their solubilization.

For example, problems of deposits of polymers frequently occur during the steps in which the monomers are dried. In conventional processes for the recovery and purification of acrylic acid and methacrylic acid, which involve a stage of absorption in water (case of the manufacture of acrylic acid by oxidation of propylene or propane, or of methacrylic acid by oxidation of isobutene or tert-butyl alcohol), the monomers are re-encountered, in an intermediate stage, as a mixture in a medium rich in water (20 to 40% water). In the case of the production of methacrylic acid by hydrolysis of methacrylamide sulphate, the crude methacrylic acid exiting from this stage comprises 1 to 5% water. As water is a good solvent for polymer chains exhibiting a relatively low degree of crosslinking, the insoluble polymers generated in the prior stages, which are dissolved in the starting medium, precipitate during the step of removal of the water (water content <0.5%) and are deposited in the reboilers and the column bottoms.

Problems of troublesome deposits can also occur when attempts are made to recover the maximum of monomer (light compound) from a mixture rich in heavy compounds obtained at the bottom of the final columns for the purification of the monomers. The enriching of the medium in heavy compounds (polymers and addition derivatives) results in the precipitation of solids, which makes it necessary to shut down the plant in order to clean it. In order to prevent this stage from being reached, it is necessary to limit the degree of recovery of monomer. As the monomer could be put to use, this results in a loss.

Likewise, the treatment of the heavy waste from the distillation of the monomers, in order to recover therefrom light fractions which can be exploited by thermal and/or catalytic cracking, results in major phenomena of fouling which are very disadvantageous industrially, which indeed even render the operation impossible in some cases.

The documents Patents EP 839 790, DE 19 851 984 and U.S. Pat. No. 4,440,625 teach the use of derivatives of alkyl- or arylsulphonic acids as antifoulants for the purpose of dispersing the acrylic polymers or the heavy addition derivatives formed during the synthesis or purification of (meth)acrylic monomers, DE 19 851 984 also envisaging the case of the tailing or cracking operations on residues from the distillation of (meth)acrylic monomers. However, the tests carried out in dynamic mode with this known family of products by the Applicant Company have not given satisfactory results from the viewpoint of industrial application.

The Applicant Company has thus sought to solve the problems of fouling due to the deposition of solid materials of polymeric origin or resulting from addition reactions to (meth)acrylic double bonds and has discovered a family of compounds soluble in the medium comprising the (meth)acrylic monomer and exhibiting the property of effectively preventing or limiting the abovementioned depositions in items of equipment for the synthesis, purification or regeneration of the (meth)acrylic monomer.

A subject-matter of the present invention is thus first the use of at least one compound of formula (I):

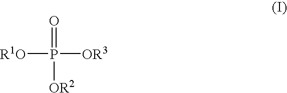

in which:
R¹ represents a C₃-C₃₀ alkyl radical, an aryl radical or an alkylaryl radical, it being possible for these radicals to be interrupted by or connected to the oxygen of the molecule via a —(OR⁴)ₒ— chain where the R⁴ groups each independently represent an ethylene, propylene or butylene chain and o is an integer from 1 to 50;
R² represents R¹, a hydrogen atom or a counterion;
R³ represents a hydrogen atom or a counterion,
   as deposition inhibitor (which can also be denoted as antifouling agent or antifoulant) which prevents the deposition of compounds of polymeric nature or resulting from addition reactions to (meth)acrylic double bonds during operations for the synthesis or purification or regeneration of (meth)acrylic monomers.

The compounds of formula (I) have a dispersing property and are also referred to subsequently as dispersants.

The compounds of formula (I) are chosen in particular from those in which R¹ and R² each independently represent the

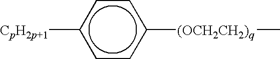

radical where p is an integer from 4 to 12, preferably 8 or 9, and q is an integer from 4 to 50, preferably 6 to 20; and R³ represents a hydrogen atom or a counterion; and/or
those in which R¹ represents the

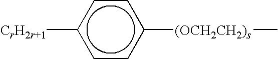

radical
where R is an integer from 4 to 12, preferably 8 or 9, and s is an integer from 4 to 50, preferably from 6 to 20; and R² and R³ each independently represent a hydrogen atom or a counterion.

Mention may be made, as counterion coming within the definition of R² and R³, of those resulting from the neutralization of the OH functional group, in the case where R² and/or R³=H, by alkanolamines and alkali metal or alkaline earth metal hydroxides. Mention may be made, as specific examples, of N⁺(CH₂CH₂OH)₃, Na⁺ and K⁺.

The compound or compounds (I) can be introduced as is into the medium comprising the (meth)acrylic monomer. They can also be introduced into the medium comprising the (meth)acrylic monomer in solution in a solvent, the said solvent being chosen in particular from aromatic solvents, such as phthalates, for example diisononyl phthalate and dioctyl phthalate, and glycol dimethyl ethers, or also in solution in the (meth)acrylic monomer.

The compound or compounds (I) can be introduced at a concentration of 0.01% to 1% by weight, in particular of 0.05 to 0.5% by weight, into the medium comprising the (meth)acrylic monomer to be prepared, purified or regenerated.

The medium comprising the (meth)acrylic monomer can also include at least one polymerization inhibitor in a proportion in particular of 0.01% to 5% by weight, especially of 0.05% to 3% by weight, with respect to the medium comprising the (meth)acrylic monomer to be prepared, purified or regenerated, it being possible for the polymerization inhibitor or inhibitors to be chosen from:
phenolic derivatives, such as hydroquinone and its derivatives, for example hydroquinone methyl ether; 2,6-di(tert-butyl)-4-methylphenol (BHT); and 2,4-di-methyl-6-(tert-butyl)phenol (Topanol A);
phenothiazine and its derivatives, such as methylene blue;
manganese salts, such as manganese acetate;
salts of thiocarbamic or dithiocarbamic acid, such as metal thiocarbamates and dithiocarbamates, for example copper di(n-butyl)dithiocarbamate;
N-oxyl compounds, such as 4-hydroxy-2,2,6,6-tetramethyl piperidinoxyl;
compounds comprising nitroso groups, such as N-nitrosophenylhydroxylamine and its ammonium salts;
quinones, such as benzoquinone;
amino compounds, such as para-phenylenediamine derivatives, represented by the general formula (II):

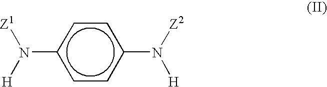

in which Z¹ and Z² each independently represent an alkyl, aryl, alkylaryl or arylalkyl radical, an example being N,N′-di(sec-butyl)-para-phenylenediamine.

The invention makes it possible to reduce depositions of solid materials in plants for the purification of (meth)acrylic monomers and thus to reduce the frequency of shutdowns necessary for the cleaning of columns, exchangers, reboilers, condensers, reactors, and the like.

A second advantage may be to reduce the amount of stabilizers introduced into the items of purification equipment, to decrease their cost.

The recovery with better yields of valuable products (monomers, reactants) in the concentration operations, or thermal or catalytic cracking operations, constitutes another advantage.

The operations affected by the present invention are very diverse and are in particular the following:
reaction stages for the synthesis of (meth)acrylic acids or esters, such as those listed in the preamble of the present description;
stages for the purification of (meth)acrylic acids or esters, in particular during the stages for separation by distillation;
stages for the generation of reactants and reaction products by thermal cracking, batchwise or continuously, in the presence or absence of catalyst.

In the case of the use according to the invention where the operation involves a distillation column, the compounds (i) are preferably introduced at the column top.

The present invention will now be described using Examples, Reference Examples and Comparative Examples.

In all these examples, the percentages are expressed by weight and the following abbreviations are used:

### Polymerization Inhibitors or Stabilizers

PTZ: phenothiazine
PMP: para-methoxyphenol
HQ: hydroquinone

### Solvent

MIBK: methyl isobutyl ketone

The dispersant used in the Examples of the invention is a neutralized mixture of ethoxylated alkylphenol mono- and diphosphate, corresponding to the definition of the formula (I) as above, in solution in an aromatic solvent.

### EXAMPLE 1 (of the Invention)

Use is made of a glass distillation device comprising a distillation column, a reboiler, a condenser and a reflux receiver.

The column, with a diameter of 38 mm, is packed with a Multiknit component made of 316L stainless steel with a height of 14 cm. The vapours are generated at the bottom in a thermosiphon reboiler heated by electrical resistances with a volume of 200 ml into which acrylic acid stabilized by 0.2% of PTZ and 0.2% of PMP is introduced. A continuous throughput of air is additionally provided into the liquid of the reboiler. The vapours are condensed at the top through a water-cooled reflux condenser and collected in a reflux receiver.

The assembly is placed under a reduced pressure of 1.6×10⁴ Pa (120 mmHg) and the temperature measured at the bottom is 89° C. The throughput of liquid condensed at the top is 540 g/h.

PMP and PTZ (10 ppm of each of the stabilizers, with respect to the throughput of distillate) and dispersant (0.3%, with respect to the throughput of distillate), in solution in acrylic acid, are continuously introduced into this receptacle.

The liquid collected in the reflux receiver is returned via a pump to the column top, a constant level being maintained in this receptacle. The level of liquid in the reboiler is kept constant by withdrawing the excess liquid.

After operating for 3 hours, the weight of polymer deposited in the Multiknit packing is 6.5 g.

### REFERENCE EXAMPLE 1

A Reference Example is carried out under the same conditions as those of Example 1 but without the dispersant: the weight of polymer recovered in the packing is 25.1 g.

### EXAMPLE 2

A mixture of acrylic acid comprising 5% of water is introduced into the same assembly as Example 1, except for the column packing, composed this time of a Multiknit packing component with a height of 4 cm and of glass Raschig rings over a height of 11 cm. Feeding with acrylic acid comprising the stabilizers PMP and PTZ and the dispersant is provided so as to convey, at the column top, 10 ppm of each of the polymerization inhibitors and 0.1% of dispersant, with respect to the throughput of distillate measured (740 g/h).

After operating for 3 hours, the weight of polymer deposited in the Multiknit packing is 4 g.

### REFERENCE EXAMPLE 2

A Reference Example is carried out under the same conditions as those in Example 2 but without the dispersant: the weight of polymer recovered in the packing is 28 g.

### COMPARATIVE EXAMPLE 2

A Comparative Example is carried out under the same conditions as those of Example 2 but with pure acrylic acid and dodecylbenzenesulphonic acid as dispersant: the weight of polymer recovered in the packing is 15.9 g.

### EXAMPLE 3

Pure methacrylic acid, replacing acrylic acid, is introduced into the assembly and under the conditions of Example 2.

No solid deposit is observed in the packing of the column.

After testing for 3 hours, in the presence of the dispersant (0.1%, with respect to the throughput of distillate), the liquid solution present in the reboiler is cloudy, with a few fine solids in suspension. After emptying the liquid, a few solid particles remain attached to the wall of the reboiler and disappear by simple washing with water.

### REFERENCE EXAMPLE 3

Under the same conditions as in Example 3 but in the absence of the dispersant, the solid particles are agglomerated into clusters, partly in suspension and partly stuck to the wall. The deposits attached to the wall, in a greater amount than Example 3 carried out with dispersant, are not removed by washing with water.

### EXAMPLES 4a AND 4b OF THE INVENTION AND REFERENCE EXAMPLES 4a AND 4b

This example describes the application of the present invention to the recovery of valuable products by thermal cracking from a stream obtained at the bottom of a column for removing the heavy compounds of a process for the manufacture of butyl acrylate.

The mixture, composed of:
10.2% of butyl acrylate;
69.6% of butyl 2-butoxypropionate;
7.2% of butyl 2-acryloyloxypropionate;
2.5% of butyl 2-hydroxypropionate;
3% of PTZ (the remainder being composed of polymers and of other heavy compounds in a lower concentration),
   is exploited with the objective of recovering the acrylic acid, the butyl acrylate and the butanol present in this mixture.

Recovery takes place in two stages: removal of the heaviest compounds (in particular the polymers) in an evaporation stage (Example 4a of the invention and Reference Example 4a), followed by cracking of the evaporated fraction in the presence of a sulphuric acid catalyst (Example 4b of the invention and Reference Example 4b).
In the first stage of the process (evaporation), a thermosiphon-circulation glass reboiler heated by electrical resistances, with a volume of 90 ml, is fed at a throughput of 360 g/h with the mixture of heavy products. The reboiler is equipped with a droplet-separating section at the top, composed of a glass component filled with a Multiknit packing with a height of 3 cm. The stream distilled at the top is condensed through a circulating-water exchanger and collected in a receiver.

The operation is carried out under a reduced pressure of 6.7×10³ Pa (50 mmHg) at a temperature of 146° C. measured in the reboiler. At the top, 189 g/h are recovered of a mixture composed of:
24.3% of butyl acrylate;
66.3% of butyl 2-butoxypropionate;
3.8% of butyl 2-acryloyloxypropionate;
2.8% of butyl 2-hydroxypropionate;
0.7% of butanol.

In a first test (Reference Example 4a), the mixture is introduced without addition of dispersant. After operating for 5 hours, the wall of the reboiler is completely covered with a solid deposit which is very difficult to remove, which makes this method of recovering in value unusable industrially.

In a second test (Example 4a of the invention), 0.04% of the dispersant is added to the feed medium and it is found that the wall of the reboiler is clean after operating for 10 hours.
In the second stage of the process (thermal catalytic cracking), the same assembly is used, the reboiler having this time a function of cracking reactor. Sulphuric acid (4% in the medium) and PTZ (1.4%) are added to the vaporizer top stream recovered during the preceding stage and then this stream is introduced at a throughput of 180 g/h into the reactor. The temperature imposed in the reactor is 186° C. and, at the top, 146 g/h are condensed of a mixture comprising:
71.3% of butyl acrylate;
1.1% of butyl 2-butoxypropionate;
0.2% of butyl 2-acryloyloxypropionate;
0.04% of butyl 2-hydroxypropionate;
14.8% of butanol and 2.5% of water.

In a first test (Reference Example 4b), the mixture is introduced without addition of dispersant. After operating for 2 hours, the wall of the reactor is covered with a solid deposit which is very difficult to remove.

In a second test (Example 4b of the invention), 0.12% of the dispersant is added to the feed medium and it is found that the wall of the reboiler is clean after operating for 6 hours.

1. A process for the synthesis or regeneration of (meth)acrylic monomers comprising adding a deposition inhibitor to the (meth)acrylic monomer stream, said deposition inhibitor comprising at least one compound of formula (I): 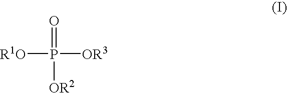
in which:
R¹ represents a C₃-C₃₀ alkyl radical, an aryl radical or an alkylaryl radical, it being possible for these radicals to be interrupted by or connected to the oxygen of the molecule via a —(OR⁴)ₒ— chain where the R⁴ groups each independently represent an ethylene, propylene or butylene chain and o is an integer from 1 to 50;
R² represents R¹, a hydrogen atom or a counterion;
R³ represents a hydrogen atom or a counterion,
wherein said deposition inhibitor prevents the deposition of compounds of polymeric nature or resulting from addition reactions to (meth)acrylic double bonds.

2. The process according to claim 1, wherein the compounds of formula (I) are chosen from those in which R¹ and R² each independently represent the 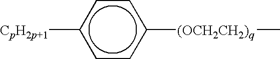
radical where p is an integer from 4 to 12, and q is an integer from 4 to 50, and R³ represents a hydrogen atom or a counterion; and/or those in which R¹ represents the 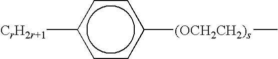
radical where R is an integer from 4 to 12, and s is an integer from 4 to 50, and R² and R³ each independently represent a hydrogen atom or a counterion.

3. The process according to claim 1, wherein the compound or compounds (i) are introduced as is into the medium comprising the (meth)acrylic monomer.

4. The process according to claim 1, wherein the compound or compounds (I) are introduced into the medium comprising the (meth)acrylic monomer in solution in a solvent or in solution in the (meth)acrylic monomer.

5. The process according to claim 4, wherein the solvent is chosen from aromatic solvents.

6. The process according to claim 1, wherein the compound or compounds (I) are introduced at a concentration of 0.01% to 1% by weight into the medium comprising the (meth)acrylic monomer to be prepared, purified or regenerated.

7. The process according to claim 6, wherein the compound or compounds (I) are introduced at a concentration of 0.05 to 0.5% by weight into the medium comprising the (meth)acrylic monomer to be prepared, purified or regenerated.

8. The process according to claim 1, wherein the medium comprising the (meth)acrylic monomer includes at least one polymerization inhibitor in a proportion of 0.01% to 5% by weight, with respect to the medium comprising the (meth)acrylic monomer to be prepared, purified or regenerated.

9. The process according to claim 8, wherein the polymerization inhibitor or inhibitors are chosen from:
phenolic derivatives;
phenothiazine and its derivatives;
manganese salts;
salts of thiocarbamic or dithiocarbamic acid;
N-oxyl compounds;
compounds comprising nitroso groups;
quinones;
amino compounds, represented by the general formula (II): 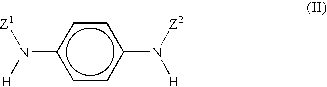
in which Z¹ and Z² each independently represent an alkyl, aryl, alkylaryl or arylalkyl radical.

10. The process according to claim 1, wherein the process comprises:
reaction stages for the synthesis of (meth)acrylic acids or esters;
stages for the purification of (meth)acrylic acids or esters, in particular during the stages for separation by distillation; and
stages for the generation of reactants and reaction products by thermal cracking, batchwise or continuously, in the presence or absence of catalyst.

11. The process according to claim 1, in which the operation involves a distillation column, characterized in that the compounds (I) are preferably introduced at the column top.

12. The process according to claim 1, wherein the (meth)acrylic monomers are chosen from acrylic acid and methacrylic acid as well as C₁-C₈ alkyl acrylates and methacrylates.

13. The process according to claim 2, wherein the compounds of formula (I) are chosen from those in which R¹ and R² each independently represent the 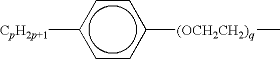
radical where p is an integer from 8 or 9, and q is an integer from 6 to 20; and R³ represents a hydrogen atom or a counterion; and/or
those in which R¹ represents the 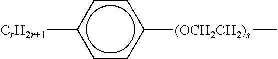
radical where R is an integer from 8 or 9, and s is an integer from 6 to 20; and R² and R³ each independently represent a hydrogen atom or a counterion.

14. The process according to claim 5, wherein the solvent is selected from the group consisting of phthalates, diisononyl phthalate, dioctyl phthalate, and glycol dimethyl ethers.

15. The process according to claim 8, wherein the medium comprising the (meth)acrylic monomer includes at least one polymerization inhibitor in a proportion of 0.05% to 3% by weight, with respect to the medium comprising the (meth)acrylic monomer to be prepared, purified or regenerated.

16. The process according to claim 9, wherein the polymerization inhibitor or inhibitors are chosen from:
hydroquinone and its derivatives selected from the group consisting of hydroquinone methyl ether; 2,6-di(tert-butyl)-4-methylphenol; and 2,4-dimethyl-6-(tert-butyl)phenol;
methylene blue;
manganese acetate;
metal thiocarbamates and dithiocarbamates;
4-hydroxy-2,2,6,6-tetramethyl piperidinoxyl;
N-nitrosophenylhydroxylamine and its ammonium salts; and
para-phenylenediamine derivatives.

17. The process according to claim 16, wherein said metal thiocarbamates and dithiocarbamates comprise copper di(n-butyl)dithiocarbamate.

18. The process according to claim 10, wherein the reaction stages for the synthesis of (meth)acrylic acids or esters, comprise
the gas-phase catalytic oxidation of propylene or propane to manufacture acrylic acid;
the catalytic oxidation of isobutene or tert-butanol to generate methacrylic acid;
the synthesis of methacrylamide sulphate from acetone cyanohydrin, methanol and sulphuric acid to produce methyl methacrylate; or
the production of (meth)acrylic esters from (meth)acrylic acid and alcohols.
